(19) United States
(12) Patent Application Publication
(10) Pub. No.: US 2009/0170958 A1
(43) Pub. Date: Jul. 2, 2009

(54) USE OF PARTICULAR POLYMERS OR COPOLYMERS AS SURFACTANTS FOR STABILIZING LATICES

(76) Inventors: Jean-Luc Couturier, Lyon (FR); Manuel Hidalgo, Brignais (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/281,353

(22) PCT Filed: Mar. 2, 2007
(86) PCT No.: PCT/EP07/52017

§ 371 Date: Oct. 15, 2008

(30) Foreign Application Priority Data
Mar. 2, 2006 (FR) 06 01876

(51) Int. Cl. C08L 13/02; A61K 47/32; C09D 11/02; C09D 133/08

(52) U.S. Cl. 514/772.4; 525/221; 525/222; 524/522; 524/523

(57) ABSTRACT

The invention relates to a composition comprising a latex and at least one copolymer that may be obtained via a monomer polymerization process, comprising a step of placing in contact, in a medium comprising water, the said monomer(s) with at least one initiator corresponding, for example, to the formula:

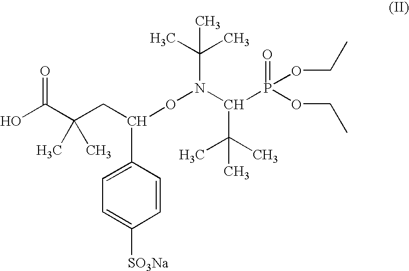

### TECHNICAL FIELD

The present invention relates to the use of particular polymers or copolymers as surfactants for stabilizing latices, in particular acrylic latices.

The general field of the invention is thus that of latices.

Latices are in the form of a composition comprising, in suspension, microscopic particles of polymer chains in a continuous liquid medium, which is usually aqueous, which may serve as a base, among other applications, for adhesives, paints, textile binders and inks (especially acrylic latices).

To ensure the stability of latices during their synthesis and over time, it is necessary to incorporate surfactants therein.

The current surfactants often generate foams, which, when they are generated during the preparation or use of the latex, may lead to the fouling of equipment.

The inventors set themselves the aim of proposing novel surfactants that afford good stabilization of latices, especially acrylic latices, and that generate little or no foam during the manufacture or use of the latices.

### DESCRIPTION OF THE INVENTION

Thus, the invention relates to a composition comprising at least one latex and at least one surfactant that may be obtained via a process of polymerizing one or more monomers, the said process comprising a step of placing in contact, for example, in a medium comprising water, the said monomer(s) with at least one initiator corresponding to formula (I) below:

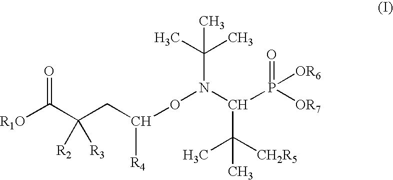

in which:
R₁ represents a hydrogen atom, a linear or branched alkyl group containing from 1 to 8 carbon atoms, a phenyl group, a metal chosen from alkali metals, alkaline-earth metals and transition metals, in particular an alkali metal (Na, Li or K), or alternatively H₄N⁺, Bu₄N⁺ or Bu₃HN⁺, Bu representing an n-butyl group;
R₂ and R₃, which may be identical or different, represent a linear or branched alkyl group containing from 1 to 3 carbon atoms;
R₅ represents a hydrogen atom or a group —OCOR₈, R₈ representing a linear or branched alkyl group containing from 1 to 20 carbon atoms;
R₆ and R₇ represent, independently, a linear or branched alkyl group containing from 1 to 3 carbon atoms;
R₄ represents:
an aryl group bearing at least one acid group comprising at least one heteroatom chosen from S and P, the said acid group possibly existing in the form of a salt; or
a heterocyclic group comprising one or more heteroatoms chosen from O, N and/or S, the said heterocyclic group optionally bearing an acid group comprising at least one heteroatom chosen from S and P or bearing at least one hydrocarbon-based group optionally comprising one or more heteroatoms (for example N, S and/or O), the said hydrocarbon-based group bearing at least one acid group as defined above, the said heterocyclic group possibly existing in the form of a salt; or
a group —CO—NR—Y or —CO—O—Y, with Y representing a hydrocarbon-based group optionally comprising one or more heteroatoms (for example N, S and/or O) bearing at least one acid group comprising a heteroatom chosen from S and P or representing a hydrocarbon-based group optionally comprising one or more heteroatoms (for example N, S and/or O) containing at least one heterocyclic group comprising one or more heteroatoms chosen from N, O and S, the said group —CO—NR—Y or —CO—O—Y possibly existing in the form of a salt and R representing a hydrogen atom or an alkyl group, preferably containing from 1 to 24 carbon atoms.

According to a first alternative, R₄ may be an aryl group comprising, for example, from 5 to 20 carbon atoms (for example a phenyl group), the said aryl group bearing (i.e. being substituted with) at least one acid group comprising at least one heteroatom chosen from S and P, the said acid group possibly existing in the form of a salt. As example of an acid group comprising at least one heteroatom chosen from S and P, mention may be made of a sulfonic, phosphonic, phosphoric or phosphinic group, and the salts thereof.

According to a second alternative, R₄ may be a heterocyclic group comprising one or more heteroatoms chosen from O, N and/or S, such as a pyrrole, pyridine, indole, thiophene, furan or pyrimidine group, optionally bearing at least one acid group comprising at least one heteroatom chosen from S and P as defined above or the said heterocyclic group bearing a hydrocarbon-based group, such as an alkyl group containing from 1 to 24 carbon atoms, optionally comprising one or more heteroatoms, the said hydrocarbon-based group being substituted one or more times with an acid group as defined above (i.e. an acid group comprising at least one heteroatom chosen from S and P).

According to a third alternative, R₄ may represent a group —CO—NR—Y or —CO—O—Y, with Y representing a hydrocarbon-based group, such as an alkyl group possibly containing from 1 to 24 carbon atoms, optionally comprising one or more heteroatoms, and substituted with at least one acid group comprising at least one heteroatom chosen from S and P, such as a sulfonic, phosphonic, phosphoric or phosphinic group. An example of such a group Y that may be mentioned is the group —C(CH₃)₂—CH₂—SO₃H. Y may also be a hydrocarbon-based group, such as an alkyl group containing from 1 to 24 carbon atoms, optionally comprising one or more heteroatoms, and bearing at least one heterocyclic group comprising one or more heteroatoms chosen from N, O and S, such as an imidazole, imidazoline, imidazolidone, pyrazole, triazole, tetrazole, thiadiazole or oxadiazole group. The group Y cannot be an unsubstituted alkyl group, insofar as it is necessarily substituted with an acid group or a heterocyclic group as defined above.

Among the initiators of formula (I), it is most particularly preferred to use those for which R₄ is an aryl group bearing at least one acid group comprising at least one heteroatom chosen from S and P, the said acid group possibly existing in the form of a salt. In particular, R₄ may advantageously be a phenylene group bearing a group —SO₃R₉, R₉ representing a hydrogen atom, a metal chosen from alkali metals, alkaline-earth metals and transition metals, in particular an alkali metal (Na, Li or K), or alternatively H₄N⁺, Bu₄N⁺ or Bu₃HN⁺, Bu representing an n-butyl group.

One particular initiator in accordance with the invention corresponds to formula (II) below:

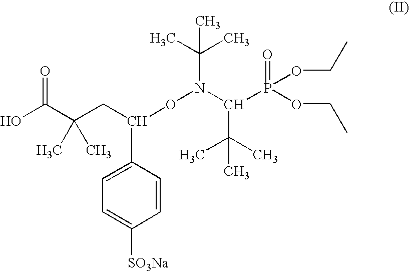

The initiators described above may be obtained by radical addition of the 1,2-type, of an olefin to a starting alkoxyamine, according to the following reaction scheme:
a) cleavage of the starting alkoxyamine into free radicals:

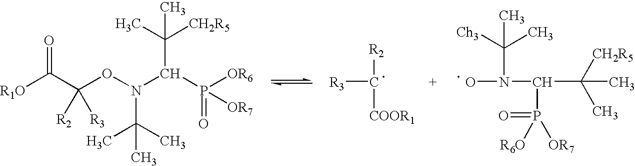

b) radical addition of the 1,2-type, of the previously formed free radicals to the olefin:

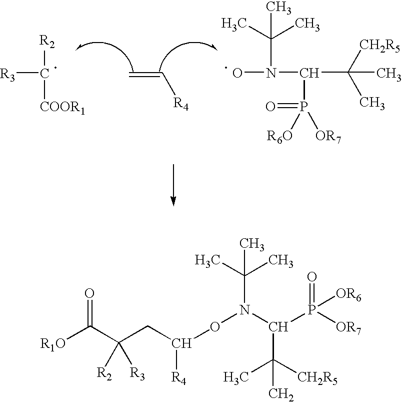

The process is advantageously performed in a medium in particular comprising water. This medium may be an aqueous or predominantly aqueous solution or an aqueous phase/organic phase dispersed medium (dispersion, emulsion, miniemulsion, microemulsion, suspension, micellar, reverse suspension, reverse emulsion or reverse microemulsion).

The surfactants present in the compositions of the invention are very advantageously in the form of an amphiphilic copolymer, i.e. a copolymer containing both hydrophilic parts and hydrophobic parts, the hydrophilic parts resulting from the polymerization of one or more hydrophilic monomers, and the hydrophobic parts resulting from the polymerization of one or more hydrophobic monomers.

It is understood that the hydrophilic monomers will be, in particular, monomers whose units after polymerization will be other than the unit —CH₂—CHR₄— present in the polymerization initiator as defined above.

The hydrophilic monomer(s) may be chosen from:
water-soluble styrene derivatives such as sodium styrenesulfonate;
water-soluble acrylic monomers such as acrylic acid and salts thereof, methyl acrylate, 2-hydroxyethyl acrylate, 2-methoxyethyl acrylate, methoxypolyethylene glycol acrylates, ethoxypolyethylene glycol acrylates, methoxypolyethylene glycol-polypropylene glycol acrylates and mixtures thereof, 2-(dimethylamino)ethyl acrylate (DMAEA), [2-(acryloyloxy)ethyl]trimethylammonium chloride or sulfate, [2-(acryloyloxy)ethyl]dimethylbenzylammonium chloride or sulfate, and alkylene glycol phosphate acrylates:
methacrylic monomers such as methacrylic acid and salts thereof, 2-hydroxyethyl methacrylate, 2-ethoxyethyl methacrylate, methoxypolyethylene glycol methacrylates, ethoxypolyethylene glycol methacrylates, methoxypolyethylene glycol-polypropylene glycol meth acrylates and mixtures thereof, 2-(dimethylamino)ethyl methacrylate (DMAEMA), [2-(methacryloyloxy)ethyl]-trimethylammonium chloride or sulfate, [2-(methacryloyloxy)ethyl]dimethylbenzylammonium chloride or sulfate, alkylene glycol phosphate methacrylates, hydroxyethylimidazolidone methacrylate, hydroxyethylimidazolidinone methacrylate and 2-(2-oxo-1-imidazolidinyl)ethyl methacrylate;
acrylamide or substituted acrylamides, N-methylolacrylamide, acrylamidopropyltrimethylammonium chloride (APTAC), acrylamidomethylpropanesulfonic acid (AMPS) and salts thereof;
methacrylamide or substituted methacrylamides, 2-methyl-N-[2-(2-oxoimidazolidinyl)-ethyl]acrylamide, N-methylolmethacrylamide, methacrylamidopropyltrimethylammonium chloride (MAPTAC);
itaconic acid, maleic acid and salts thereof, maleic anhydride, alkyl or alkoxy- or aryloxypolyalkylene glycol maleates or hemimaleates, vinylpyridine, vinylpyrrolidinone; and
a mixture of at least two of the abovementioned monomers.

Water-soluble monomers that are particularly advantageous in the context of this invention are:
(meth)acrylic acid and salts thereof;
(meth)acrylates of amine salts, such as [2-(methacryloyloxy)ethyl]trimethylammonium chloride or sulfate, [2-(methacryloyloxy)ethyl]dimethylbenzylammonium chloride or sulfate;
hydroxyalkyl (meth)acrylates, such as 2-hydroxyethyl methacrylate;
polyethylene glycol, alkoxy- or aryloxypolyalkylene glycol (meth)acrylates, such as methoxypolyethylene glycol (meth)acrylates or ethoxypolyethylene glycol (meth)acrylates; and
mixtures thereof.

The hydrophobic monomer(s) may be chosen from:
vinylaromatic monomers such as styrene or α-methylstyrene;
diene monomers, such as butadiene or isoprene;
hydrophobic acrylate monomers, such as ethyl acrylate, n-butyl acrylate, ethylhexyl acrylate or phenyl acrylate, methoxypolypropylene glycol acrylates, fluoro acrylates or silyl acrylates;
methacrylate monomers, such as methyl methacrylate, lauryl methacrylate, cyclohexyl methacrylate, allyl methacrylate or phenyl methacrylate, methoxypolypropylene glycol methacrylates, 2-(tert-butylamino)ethyl methacrylate (TBAEMA), fluoro methacrylates, such as 2,2,2-trifluoroethyl methacrylate, and silyl methacrylates such as 3-methacryloyl-propyltrimethylsilane;
acrylonitrile; and

mixtures thereof.

A surfactant in accordance with the invention is, for example, a copolymer comprising hydrophilic units derived from the polymerization of methacrylic acid and hydrophobic units derived from the polymerization of methyl methacrylate.

The initiator(s) of formula (I) may be present in a content ranging from 0.005% to 40% and preferably in a content ranging from 0.01% to 10% by weight relative to the total weight of the monomer(s) used. The monomer(s) and/or the initiator of alkoxyamine type may optionally be introduced continuously into the polymerization medium.

According to one embodiment of the invention, the step of placing in contact may be performed, in addition to the initiator of formula (I), in the presence of an initiator chosen from hydroperoxides, dialkyl peroxides, diacyl peroxides, peroxy esters, peroxydicarbonates, peroxyacetals and azo compounds.

Examples of hydroperoxides that may be mentioned include tert-butyl hydroperoxide, tert-amyl hydroperoxide, cumene hydroperoxide, 2,5-dimethyl-2,5-di(hydroperoxy)hexane, diisopropylbenzene monohydroperoxide and para-menthane hydroperoxide.

Examples of dialkyl peroxides that may be mentioned include 2,5-dimethyl-2,5-di(tert-butylperoxy)hexyne-(3), di-tert-butyl peroxide, di-tert-amyl peroxide, 1,3-di(tert-butylperoxyisopropyl)benzene, 2,5-dimethyl-2,5-di(tert-butylperoxy)hexyne, 1,1,4,4,7,7-hexamethylcyclo-4,7-diperoxynonane and 3,3,3,6,6,9,9-hexamethylcyclo-1,2,4,5-tetraoxanonane.

Examples of diacyl peroxides that may be mentioned include benzoyl peroxide, lauroyl peroxide, decanoyl peroxide, 3,5,5-trimethylhexanoyl peroxide, and acetylcyclohexylsulfonyl peroxide.

Examples of peroxy esters that may be mentioned include tert-butyl peroxybenzoate, tert-butyl peroxyacetate, tert-butyl peroxy-3,5,5-trimethylhexanoate, tert-amyl peroxy-3,5,5-trimethylhexanoate, 2,5-dimethyl-2,5-di(benzoylperoxy)hexane, 00-tert-butyl-O-isopropyl monoperoxycarbonate, 00-tert-butyl-O-(2-ethylhexyl) monoperoxycarbonate, 00-tert-amyl-O-(2-ethylhexyl) monoperoxycarbonate, tert-butyl peroxyisobutyrate, tert-butyl peroxy-2-ethylhexanoate, tert-amyl peroxy-2-ethylhexanoate, 2,5-dimethyl-2,5-bis(2-ethylhexanoylperoxy)hexane, tert-butyl peroxyneodecanoate, tert-butyl peroxyisononanoate, tert-butyl peroxypivalate, tert-amyl peroxypivalate, α-cumyl peroxyneodecanoate, tert-amyl peroxydecanoate, tert-butyl 3-hydroxy-1,1-dimethylbutylperoxyneodecanoate and tert-butyl peroxymaleate.

Examples of peroxydicarbonates that may be mentioned include di(2-ethylhexyl) peroxydicarbonate, dicyclohexyl peroxydicarbonate, di(n-propyl) peroxydicarbonate and di(4-tert-butylcyclohexyl) peroxydicarbonate.

Examples of peroxyacetals that may be mentioned include 1,1-di(tert-butylperoxy)cyclohexane, 1,1-di(tert-butylperoxy)-3,3,5-trimethylcyclohexane, ethyl 3,3-di(tert-butylperoxy)butyrate, ethyl 3,3-di(tert-amylperoxy)butyrate, n-butyl 4,4-di(tert-butylperoxy)valerate, 2,2-di(tert-butylperoxy)butane, 1,1-di(tert-amylperoxy)cyclohexane and 2,2-bis[4,4-di(tert-butylperoxy)cyclohexyl]propane.

The step of placing in contact may also be performed in the presence of mineral oxidizing agents such as sodium, potassium or ammonium persulfates, hydrogen peroxide, perchlorates, percarbonates or ferric salts. These oxidizing agents may be used alone or in combination with mineral or organic reducing agents such as sodium or potassium bisulfite or metabisulfite, vitamin C, and sodium or potassium hypophosphite. These organic or mineral reducing agents may also be used alone, i.e. in the absence of mineral oxidizing agents.

The process of the invention may include a step of in situ preparation of the initiator of formula (I), preferably before the step of placing in contact.

From a structural point of view, the surfactants, besides the repeating units derived from the polymerization of the monomers, contain at least one reactive end having the following formula:

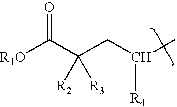

R₁, R₂, R₃ and R₄ being as defined above.

The polymers or copolymers will in particular contain a unit of formula —CH₂—CHR₄— derived from the alkoxyamine present at the end of the chains, the other units resulting from the polymerization of the monomers, which units will be different from the said unit —CH₂—CHR₄—.

This polymer or copolymer containing such a reactive end may be made to undergo a chemical trans-formation of this end by reacting it with suitable reagents.

The latices present in the compositions of the invention may be latices obtained by emulsion polymerization, in particular latices of alkali-swellable emulsion type, more commonly known as ASE latices.

Examples of ASE latices that may be mentioned include:
the latex obtained by emulsion polymerization in aqueous medium of methacrylic acid, ethyl acrylate and diallyl phthalate;
the latex obtained by emulsion polymerization in aqueous medium of methacrylic acid, styrene and ethylene glycol dimethacrylate;
the latex obtained by emulsion polymerization in aqueous medium of methacrylic acid, methyl methacrylate and diallyl phthalate.

These latices have the characteristic of having thickening properties.

These compositions may be prepared by mixing the surfactant copolymer with the precursor monomers of the latex, followed by a step of polymerization of the said monomers. The surfactant may be prepared in situ prior to the addition of the monomers, as may the initiator required for the synthesis of the surfactant.

The compositions in accordance with the invention may be used in any field using latices, namely in the field of paints, textiles, cosmetics, paper, floorcoverings, construction and decoration.

More particularly, the compositions of the invention may be used as dispersants in cosmetic compositions, paint compositions and ink compositions.

Finally, according to a second subject, the invention relates to the use, as a latex-stabilizing surfactant, of a polymer or copolymer that may be obtained via a process of polymerization of one or more monomers, comprising a step of placing in contact, in a medium comprising water, the said monomer(s) with at least one initiator corresponding to formula (I) below:

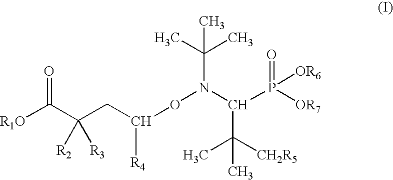

in which:
R₁ represents a hydrogen atom, a linear or branched alkyl group containing from 1 to 8 carbon atoms, a phenyl group, a metal chosen from alkali metals, alkaline-earth metals and transition metals, in particular an alkali metal (Na, Li or K) or alternatively H₄N⁺, Bu₄N⁺ or Bu₃HN⁺, Bu representing an n-butyl group;
R₂ and R₃, which may be identical or different, represent a linear or branched alkyl group containing from 1 to 3 carbon atoms;
R₅ represents a hydrogen atom or a group —OCR₈,

R₈ representing a linear or branched alkyl group containing from 1 to 20 carbon atoms;
R₆ and R₇ represent, independently, a linear or branched alkyl group containing from 1 to 3 carbon atoms;
R₄ represents:
an aryl group bearing at least one acid group comprising at least one heteroatom chosen from S and P, the said acid group possibly existing in the form of a salt; or
a heterocyclic group comprising one or more heteroatoms chosen from O, N and/or S, the said heterocyclic group optionally bearing at least one acid group comprising at least one heteroatom chosen from S and P or bearing a hydrocarbon-based group optionally comprising one or more heteroatoms (for example N, S and/or O), the said hydrocarbon-based group bearing at least one acid group as defined above, the said heterocyclic group possibly existing in the form of a salt; or
a group —CO—NR—Y or —CO—O—Y, with Y representing a hydrocarbon-based group optionally comprising one or more heteroatoms (for example N, S and/or O) bearing at least one acid group comprising a heteroatom chosen from S and P or representing a hydrocarbon-based group optionally comprising one or more heteroatoms (for example N, S and/or O) containing at least one heterocyclic group comprising one or more heteroatoms chosen from N, O and S, the said group —CO—NR—Y or —CO—O—Y possibly existing in the form of a salt and R representing a hydrogen atom or an alkyl group, preferably containing from 1 to 24 carbon atoms.

The polymers or copolymers that may be used in this sense are the same as those described above for the compositions of the invention.

The invention will now be described with reference to the following examples, which are given as non-limiting illustrations.

### DETAILED DESCRIPTION OF PARTICULAR EMBODIMENTS

### Example 1

This example illustrates the preparation of an initiator of formula (II) below:

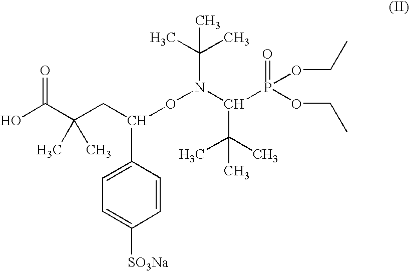

starting with an alkoxyamine having the following formula:

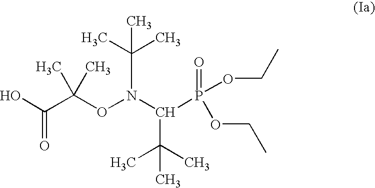

and sodium 4-styrenesulfonate:

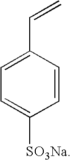

1 litre of ethanol and 0.5 litre of degassed water are placed in a 2-litre glass reactor purged with nitrogen. 54 g of sodium 4-styrenesulfonate (0.262 mol) and 100 g of alkoxyamine of formula (Ia) (0.262 mol) are added. The mixture is heated to 70° C. and left to react for 6 hours with stirring. The product is recovered by evaporation under vacuum at a temperature of 30° C. 179 g of a yellow oil that crystallizes on storage at 4° C. in the form of a wax are obtained.

The product obtained is analysed by ¹H, ¹³C and ³¹P NMR, by negative electrospray mass spectrometry and by Karl Fischer analysis. It contains 84% by weight of initiator of formula (II) in the form of two diastereoisomers in 59/41 proportions and 16% water.

The characteristics of the initiator obtained are as follows:

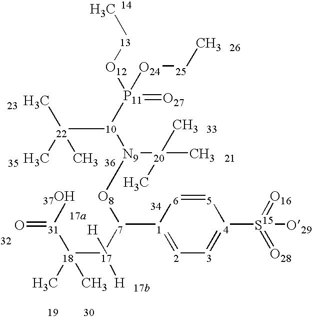

¹H NMR (CDCl₃)

Major isomer

| Atom No. | Chemical shift (in ppm) |
|---|---|
| 2 | 7.45 |
| 3 | 7.7 |
| 5 | 7.7 |
| 6 | 7.45 |
| 7 | 5.1 |
| 19 | 1.13 |
| 30 | 1.13 |
| 10 | 3.4 |
| 17a | 2.3 |
| 17b | 2.5 |
| 21 | 0.8 |
| 23 | 1.10-1.21 |
| 33 | 0.8 |
| 34 | 0.8 |
| 35 | 1.10-1.21 |
| 36 | 1.10-1.21 |
| 25 | 3.2 |
| 13 | 3.65-4.45 |
| 26 | 0.9 |
| 14 | 1.20-1.40 |

Minor isomer

| Atom No. | Chemical shift (in ppm) |
|---|---|
| 7 | 4.9 |
| 6 | 7.3 |
| 2 | 7.3 |
| 5 | 7.7 |
| 3 | 7.7 |
| 10 | 3.32 |
| 23 | 1.10-1.22 |
| 35 | 1.10-1.22 |
| 36 | 1.10-1.22 |
| 26 | 0.9 |
| 13 | 3.65-4.45 |
| 14 | 1.20-1.40 |
| 17 | 2.50-2.75 |
| 19 | 1.1 |
| 21 | 1.19 |
| 25 | 3.65-4.45 |
| 30 | 1.1 |
| 33 | 1.19 |
| 34 | 1.19 |

¹³C NMR (CDCl₃)

Major isomer

| Atom No. | Chemical shift (in ppm) |
|---|---|
| 1 | 141.60-143.40 |
| 2 | 129.50 |
| 3 | 125.00 |
| 4 | 141.60-143.40 |
| 5 | 125.00 |
| 6 | 129.50 |
| 7 | 79.80 |
| 10 | 69.50 |
| 13 | 61.50 |
| 14 | 15.95-16.50 |
| 17 | 43.00 |
| 18 | 39.72 |
| 19 | 22.30 |
| 20 | 61.20 |
| 21 | 28.20 |
| 22 | 35.00 |
| 23 | 30.20 |
| 25 | 58.70 |
| 26 | 15.95-16.50 |
| 30 | 22.30 |
| 31 | 179.80 |
| 33 | 28.20 |
| 34 | 28.20 |
| 35 | 30.20 |
| 36 | 30.20 |

Minor isomer

| Atom No. | Chemical shift (in ppm) |
|---|---|
| 1 | 141.60-143.40 |
| 2 | 128.80 |
| 3 | 124.60 |
| 4 | 141.60-143.40 |
| 5 | 124.60 |
| 6 | 128.80 |
| 7 | 86.60 |
| 10 | 69.40 |
| 13 | 61.50 |
| 14 | 15.95-16.50 |
| 17 | 45.20 |
| 18 | 39.69 |
| 19 | 21.80 |
| 20 | 60.90 |
| 21 | 28.20 |
| 22 | 35.30 |
| 23 | 30.20 |
| 25 | 58.70 |
| 26 | 15.95-16.50 |
| 30 | 21.80 |
| 31 | 179.80 |
| 33 | 28.20 |
| 34 | 28.20 |
| 35 | 30.20 |
| 36 | 30.20 |

³¹P NMR (CDCl₃)

Major isomer: 24 ppm
Minor isomer: 25 ppm

### Example 2

The example below relates to the preparation of a surfactant based on methacrylic acid and methyl methacrylate.

430 g of demineralized water are introduced into a 1-litre glass reactor equipped with a variable-speed stirring motor, inlets for the introduction of reagents, lines for the introduction of inert gases for stripping out oxygen, for instance nitrogen, and measuring probes, a system of vapour condensation with reflux, and a jacket for heating/cooling the contents of the reactor by means of circulation therein of a heat-exchange fluid; the stirring is started at moderate speed, the heating system is switched on to reach 50° C. in the reactor, and the system is degassed by bubbling nitrogen into the liquid for at least 15 minutes.

The following are separately prepared, in suitable containers:
1) a mixture of 1.38 g of methacrylic acid and 1.6 g of methyl methacrylate;
2) an aqueous solution containing 1.82 g of the initiator (II) (purity of 84% by weight) and 20 g of demineralized water.

All of solution 2 is added to the reactor and the stirring speed is set at 344 rpm. Next, mixture 1 is added to the reactor using a metering pump, over a period of 20 minutes. The reactor is then maintained at a temperature of at least 50° C. for a duration of at least 4 hours. At the end of this temperature maintenance period, the medium is cooled and the contents of the reactor are discharged into a suitable container. The product thus obtained has a milky and slightly foamy appearance, with a particle size of 169 nm measured by quasi-elastic light scattering with a Malvern LoC machine.

### Example 3

The following example illustrates the synthesis of a latex of the ASE type stabilized with a surfactant polymer.

303 g of the product prepared according to Example 2, consisting of a dilute aqueous dispersion of a surfactant polymer according to the invention, are introduced into a 1-litre glass reactor equipped with a variable-speed stirring motor, inlets for the introduction of reagents, lines for the introduction of inert gases for stripping out oxygen, for instance nitrogen, and measuring probes (for example temperature probes), a system of vapour condensation with reflux, and a jacket for heating/cooling the contents of the reactor by means of circulation therein of a heat-exchange fluid. Stirring is started at moderate speed, the heating system is switched on to reach 72° C. in the reactor, and the system is degassed by bubbling nitrogen into the liquid for at least 15 minutes.

The following are separately prepared, in suitable containers:
1) a mixture comprising 41.68 g of methacrylic acid, 90 g of ethyl acrylate and 0.40 g of diallyl phthalate; and
2) an aqueous solution comprising 0.16 g of ammonium persulfate and 5 g of demineralized water.

The reactor is brought to 78° C. and all of solution 2 is added to the reactor while stirring at a speed of 344 rpm. Seven minutes after the introduction of solution 2), mixture 1 is metered into the reactor using a metering pump, so as to complete the addition of this mixture to the reactor in 2.5 hours, during which time the temperature is maintained at least 78° C. After the addition, the medium is maintained at at least 78° C. for at least a further 3 hours. The reactor is then cooled and its contents are poured into a suitable container. The product obtained is a stable latex of the ASE type with a solids content of 27.6%, measured by gravimetry, and a particle size of 124 nm, measured with a Malvern LoC machine.

In order to confirm the thickening power of the latex of the ASE type, an aqueous dispersion with a solids content of 3% is prepared by diluting some of the product obtained with demineralized water (for example 38 g of latex with a solids content of 27.6% diluted with 312 g of demineralized water). The pH of the diluted latex is then 3.5, measured by potentiometry, and its viscosity is very close to that of water. 36% sodium hydroxide solution is then added slowly with stirring of the latex, so as to obtain alkaline swelling and the thickening effect. The maximum viscosity is obtained at about pH 7.4, at which value the dispersion at 3% takes the form of a translucent, virtually transparent aqueous gel with a viscosity of 8826 mPa·s at 30 rpm, measured with a variable spin-speed Brookfield viscometer. The gel moreover has coloured tints ranging from red to blue passing through green, depending on the angle of observation. These optical phenomena may be observed on gels obtained using the latex of the ASE type prepared in the absence of standard surfactants.

### Example 4

In order to make the manufacture of the latex of Example 3 more productive, its synthesis may be performed in the continuation of that of the surfactant polymer of Example 2. To do this, the total amount of product of Example 2 may be adjusted so as to have only 303 g, and the step of maintenance at 50° C. after the addition of the monomers of Example 2 may be limited to a minimum of 1 or 2 hours, after which the reactor may be brought to 78° C. to follow on with the operations of Example 3.

### Example 5

In order to make the manufacture of the latex of Example 4 more productive, its synthesis may be performed in the continuation of that of the water-soluble initiator of Example 1. To do this, the synthesis of the water-soluble initiator of Example 1 may be performed in the polymerization reactor, by adjusting the dilution rate with water and a small amount of ethyl alcohol, and also the amounts of sodium 4-styrenesulfonate and of alkoxyamine (Ia), so as to obtain only the amount of water-soluble initiator (II) necessary for the preparation of the surfactant of Example 2. The heating time at 70° C. for the preparation of the water-soluble initiator may be optimized by reducing it to a minimum of 1 hour, after which period the operations of Example 4 may follow on. Advantageously, this method does not require the separation and/or purification of the initiator (II) or its handling in the form of wax, since it is produced directly in the polymerization reactor (in dilute solution and in the required amount) in which it will be used for the manufacture of the surfactant polymer, which will, in turn, be used for the manufacture of the final ASE latex.

1. Composition comprising at least one latex and at least one surfactant that may be obtained via a monomer polymerization process, comprising a step of placing the said monomer(s) in contact with at least one initiator corresponding to formula (I) below: 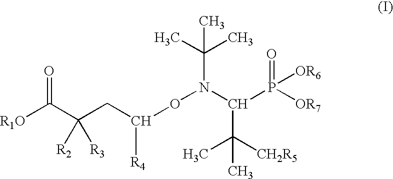
in which:
R₁ represents a hydrogen atom, a linear or branched alkyl group containing from 1 to 8 carbon atoms, a phenyl group, a metal chosen from alkali metals, alkaline-earth metals and transition metals, H₄N⁺, Bu₄N⁺ or Bu₃HN⁺, Bu representing an n-butyl group;
R₂ and R₃, which may be identical or different, represent a linear or branched alkyl group containing from 1 to 3 carbon atoms;
R₅ represents a hydrogen atom or a group —OCOR₈, R₈ representing a linear or branched alkyl group containing from 1 to 20 carbon atoms;
R₆ and R₇ represent, independently, a linear or branched alkyl group containing from 1 to 3 carbon atoms;
R₄ represents:
an aryl group bearing at least one acid group comprising at least one heteroatom chosen from S and P, the said acid group possibly existing in the form of a salt; or
a heterocyclic group comprising one or more heteroatoms chosen from O, N and/or S, the said heterocyclic group optionally bearing at least one acid group comprising at least one heteroatom chosen from S and P or bearing a hydrocarbon-based group optionally comprising one or more heteroatoms, the said hydrocarbon-based group bearing at least one acid group as defined above, the said heterocyclic group possibly existing in the form of a salt; or
a group —CO—NR—Y or —CO—O—Y, with Y representing a hydrocarbon-based group optionally comprising one or more heteroatoms bearing at least one acid group comprising a heteroatom chosen from S and P or representing a hydrocarbon-based group optionally comprising one or more heteroatoms containing at least one heterocyclic group comprising one or more heteroatoms chosen from N, O and S, the said group —CO—NR—Y or —CO—O—Y possibly existing in the form of a salt and R representing a hydrogen atom or an alkyl group.

2. Composition according to claim 1, in which R₄ is an aryl group bearing at least one sulfonic, phosphonic, phosphoric or phosphinic group, these groups possibly existing in the form of salts.

3. Composition according to claim 1, in which R₄ is a pyrrole, pyridine, indole, thiophene, furan or pyrimidine group.

4. Composition according to claim 1, in which R₄ represents a group —CO—NR—Y or —CO—O—Y, with Y representing a hydrocarbon-based group bearing at least one imidazole, imidazoline, imidazolidone, pyrazole, triazole, tetrazole, thiadiazole or oxadiazole group.

5. Composition according to claim 1, in which R₄ is a phenylene group bearing a group —SO₃R₉, R₉ representing a hydrogen atom or a metal chosen from alkali metals, alkaline-earth metals and transition metals, H₄N⁺, Bu₄N⁺ or Bu₃HN⁺, Bu representing an n-butyl group.

6. Composition according to claim 5, in which the initiator corresponds to formula (II) below:

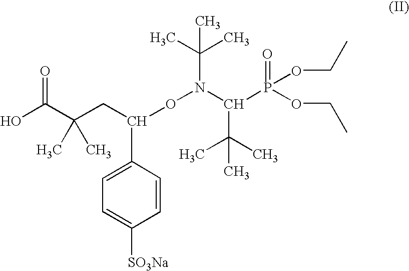

7. Composition according to claim 1, in which the copolymer is an amphiphilic copolymer, comprising repeating units derived from the polymerization of one or more hydrophilic monomers and repeating units derived from the polymerization of one or more hydrophobic monomers.

8. Composition according to claim 7, in which the hydrophilic monomer(s) is (are) chosen from:
(meth)acrylic acid and salts thereof;
(meth)acrylates of amine salts;
hydroxyalkyl (meth)acrylates;
polyethylene glycol, alkoxy- or aryloxy-polyalkylene glycol (meth)acrylates; and
mixtures thereof.

9. Composition according to claim 7, in which the hydrophobic monomer(s) is (are) chosen from:
vinylaromatic monomers such as styrene or α-methylstyrene;
diene monomers, such as butadiene or isoprene;
hydrophobic acrylate monomers, such as ethyl acrylate, n-butyl acrylate, ethylhexyl acrylate or phenyl acrylate, methoxypolypropylene glycol acrylates, fluoro acrylates or silyl acrylates;
methacrylate monomers, such as methyl methacrylate, lauryl methacrylate, cyclohexyl methacrylate, allyl methacrylate or phenyl methacrylate, methoxypolypropylene glycol methacrylates, 2-(tert-butylamino)ethyl methacrylate (TBAEMA), fluoro methacrylates, such as 2,2,2-trifluoroethyl methacrylate, and silyl methacrylates such as 3-methacryloyl-propyltrimethylsilane;
acrylonitrile; and
mixtures thereof.

10. Composition according to claim 1, in which the latex is an ASE latex.

11. Composition according to claim 1, in which the latex is chosen from:
latices obtained by emulsion polymerization in aqueous medium of methacrylic acid, ethyl acrylate and diallyl phthalate;
latices obtained by emulsion polymerization in aqueous medium of methacrylic acid, styrene and ethylene glycol dimethacrylate;
latices obtained by emulsion polymerization in aqueous medium of methacrylic acid, methyl methacrylate and diallyl phthalate.

12. A method of stabilizing a polymer or copolymer that may be obtained via a process of polymerization of one or more monomers, comprising placing said monomer(s) in contact with at least one initiator corresponding to formula (I) below: 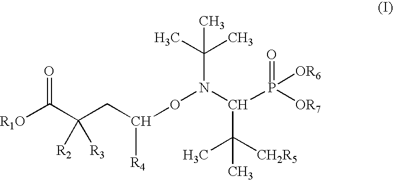
in which:
R₁ represents a hydrogen atom, a linear or branched alkyl group containing from 1 to 8 carbon atoms, a phenyl group, a metal chosen from alkali metals, alkaline-earth metals and transition metals, H₄N⁺, Bu₄N⁺ or Bu₃HN⁺, Bu representing an n-butyl group;
R₂ and R₃, which may be identical or different, represent a linear or branched alkyl group containing from 1 to 3 carbon atoms;
R₅ represents a hydrogen atom or a group —OCOR₈, R₈ representing a linear or branched alkyl group containing from 1 to 20 carbon atoms;
R₆ and R₇ represent, independently, a linear or branched alkyl group containing from 1 to 3 carbon atoms;
R₄ represents:
an aryl group bearing at least one acid group comprising at least one heteroatom chosen from S and P, the said acid group possibly existing in the form of a salt; or
a heterocyclic group comprising one or more heteroatoms chosen from O, N and/or S, the said heterocyclic group optionally bearing at least one acid group comprising at least one heteroatom chosen from S and P or bearing a hydrocarbon-based group optionally comprising one or more heteroatoms, the said hydrocarbon-based group bearing at least one acid group as defined above, the said heterocyclic group possibly existing in the form of a salt; or
a group —CO—NR—Y or —CO—O—Y, with Y representing a hydrocarbon-based group optionally comprising one or more heteroatoms bearing at least one acid group comprising a heteroatom chosen from S and P or representing a hydrocarbon-based group optionally comprising one or more heteroatoms containing at least one heterocyclic group comprising one or more heteroatoms chosen from N, O and S, the said group —CO—NR—Y or —CO—O—Y optionally existing in the form of a salt and R representing a hydrogen atom or an alkyl group.

13. A method according to claim 12, in which the copolymer is an amphiphilic copolymer, comprising repeating units derived from the polymerization of one or more hydrophilic monomers and repeating units derived from the polymerization of one or more hydrophobic monomers.

14. In a cosmetic compositions, paint compositions or ink compositions comprising a dispersant, the improvement wherein the dispersant is a composition according to claim 1.
